(19) United States
(12) Patent Application Publication
(10) Pub. No.: US 2026/0298935 A1
(43) Pub. Date: Oct. 1, 2026

(54) METHOD FOR DETECTING AND QUANTIFYING EXOSOMES

(71) Applicant: THERAWIS DIAGNOSTICS GMBH, München (DE)

(72) Inventors: Olaf WILHELM, München (DE); Evelyn HAMMER, Wasserburg am Inn (DE); Rudolf NAPIERALSKI, München (DE)

(21) Appl. No.: 19/477,914

(22) PCT Filed: Apr. 24, 2024
(86) PCT No.: PCT/EP2024/061326

§ 371 Date: Oct. 23, 2025

(30) Foreign Application Priority Data
Apr. 25, 2023 (EP) 23169918.2

(51) Int. Cl. G01N 33/575; G01N 15/1429; G01N 33/569; G01N 33/58

(52) U.S. Cl. CPC G01N 33/5758; G01N 15/1429; G01N 33/56983; G01N 33/582; G01N 2333/165; G01N 2469/10

(57) ABSTRACT

The present invention relates to a method for detecting and/or quantifying exosomes in a sample. The method of the invention comprises the steps of: providing a sample that is known to comprise or suspected of comprising exosomes; contacting a binding agent that specifically binds to the membrane of extracellular vesicles with the sample; and detecting and/or quantifying exosomes by flow cytometry and/or super-resolution microscopy, fluorometer, luminometer and optionally isolating exosomes. The binding agent comprises a label and a phosphatidylethanolamine-binding lantibiotic. The present invention further pertains to methods for detecting and/or diagnosing cancer and virus infections, methods for monitoring tumor growth or virus diseases, methods for quantifying and/or qualifying tumor-related exosomes, and to kits comprising binding agents and instructions for carrying out the methods.

### FIELD OF THE INVENTION

The present invention relates to the field of exosomes and in particular to a method for detecting and quantifying exosomes in a sample using a binding agent that specifically binds to phosphatidylethanolamine in the membrane of exosomes. The present invention further pertains to methods for detecting and/or diagnosing cancer and virus infections, methods for monitoring tumor growth or virus diseases, methods for quantifying and/or qualifying tumor-related exosomes, and to kits comprising binding agents and instructions for carrying out the methods.

### BACKGROUND OF THE INVENTION

Exosomes have first been reported in 1983 when culturing immature red blood cells with labeled transferrin receptors to trace the movement of the transferrin receptors from plasma membranes into the reticulocytes. It was observed that the labeled transferrin receptors were internalized within the reticulocytes, and then repackaged into small vesicles inside them (Harding 1983; Pan 1983). These vesicles were later termed “exosomes” (Johnstone 1989).

Exosomes belong to a large family of membrane vesicles referred to as extracellular vesicles (EVs), which generally include microvesicles (approx. 100-1,000 nm in diameter), apoptotic blebs (approx. 500-1,000 nm in diameter), and exosomes (approx. 30-150 nm in diameter) (Li 2017). Exosomes are thus the smallest type of extracellular microvesicles and are produced in inward budding multivesicular bodies (MVB) resulting in intra-luminal vesicles (ILV). If an MVB fuses with the cell surface (the plasma membrane), these ILVs are released as exosomes by exocytosis into the extracellular media. Exosomes can also be produced by the Golgi apparatus. These exosomes will be released through late endosomes (McAndrews and Kallun 2019). Exosomes have a characteristic lipid bilayer which has an average thickness of about 5 nm. The lipid components of exosomes include ceramide, cholesterol, sphingolipids, and phosphoglycerides with long and saturated fatty-acyl chains. The outer surface of exosomes is rich in saccharide chains, such as mannose, polylactosamine, alpha-2,6 sialic acid, and N-linked glycans (summarized in Li 2017).

In addition to performing many biological functions, particularly cell-cell communication, cumulative evidence has suggested that several biological entities in exosomes like proteins and microRNAs are closely associated with the pathogenesis of most human malignancies (Li 2017).

As stated above, exosomes are generated in various compartments of the cell, e.g. endosomes and Golgi apparatus. The different processes leading to the generation of exosomes are regulated by Rab GTPases. Among these GTPases, Rab4 seems to be involved in the recycling of exosomes from early endosomes, and Rab11 regulates the slow transport from perinuclear recycling endosome compartment. Rab11 is further proposed to regulate the transport of microvesicle endosomes to the plasma membrane and thus to exosome release (Blanc and Vidal 2018). It has been shown that overexpression of Rab11 stimulates the exosome release in K562 cells, and the inhibition of Rab11 function decreases exosome release (Savina 1997).

Secretion of exosomes occurs from normal (thrombocytes, immune cells, etc.) and tumor cells. They can carry DNA, RNA, microRNA, proteins, and lipids. Exosome-associated proteins used as biomarkers today include tetraspanins (CD9, CD63, CD81), immune regulation molecules (HLA-G, MHCI/II), membrane transport and fusion proteins (Rab5). The molecular composition of exosomes is assumed to reflect the (patho-) physiological changes in their cell or tissue of origin (Jia 2017).

Viruses enter cells through the endocytic pathway. Viruses that enter through endocytosis can hijack and use exosomal pathways for their own benefit. Exosomes have several characteristics that are like some viruses. These characteristics include biogenesis, uptake by cells, and intercellular transfer of functional RNAs, mRNAs, and proteins. Virus-infected cells have been shown to secrete exosomes that vary from their viral counterparts but may comprise of viral RNAs and viral proteins (Crenshaw 2018). Such exosomes are referred to as “virosomes”. Thus, identification of exosomes released from cells upon viral infection (virosomes), and identification of viral proteins comprised in said virosomes will allow diagnosing virus infection with high sensitivity and independent of DNA or RNA analysis.

However, the isolation, enrichment and detection of exosomes has proven to be complicated (van der Pol 2012; Thind 2016; Jia 2016). Due to the complexity of body fluids, physical separation of exosomes from cells and similar-sized particles turned out to be challenging. Methods have been applied to isolate exosomes such as immune-capturing with antibodies, ultracentrifugation, and precipitation with PEG6000, water deprivation etc. The isolation of exosomes using differential ultracentrifugation was found to result in co-isolation of proteins and other contaminants and incomplete separation of vesicles from lipoproteins. Combining ultracentrifugation with micro-filtration or a gradient was suggested to improve purity (Tauro 2012; van Deun 2014). Further, a single step isolation of extracellular vesicles by size-exclusion chromatography has been demonstrated to provide greater efficiency for recovering intact vesicles over centrifugation (Boing 2014), although a size-based technique alone will not be able to distinguish exosomes from other vesicle types. In addition, when applying these conventional methods, a mixed population of exosomes of different intracellular origin and further vesicles are generally co-purified.

Aggregation of EVs through antibodies directed against certain marker molecules has been employed to study the phenotype of these particles by flow cytometry, or alternatively ultracentrifugation followed by immunoblot analysis (Willms 2016). However, such bulk analysis did not allow the study of individual EVs (Willms 2016), and thus, the information conveyed through EVs, e.g. mirroring the state of the donor cell, becomes lost. This has severely hampered the ability to study the use of EVs in detection of defined changes within cells, for example after a viral infection or cancerous disease.

Commercially available exosome enrichment and/or isolation kits include the “Total Exosome Isolation Reagent” from Invitrogen (distributed by ThermoFisher Scientific), the “Exo-spin kit” from Cell Guidance Systems, the “exoEasy Maxi Kit” from Qiagen, and the Exosome Isolation Pan Kit from Miltenyi Biotec.

WO 2019/099955 discloses a method for isolating exosomes from a cultivated placenta using affinity chromatography with binding agents against inter alia a Rab family GTPase.

WO 2021/209622 discloses a method for enriching exosomes, which method targets the extra-vesicular part of Rab4 and/or Rab11 on the exosomal membrane for identifying and isolating exosomes.

However, albeit progress has been made in the field, the known methods do not satisfyingly detect and quantify essentially all exosomes in a sample, but rather fractions or subpopulation of the exosomes contained in such sample. There is, thus, a long felt need in the field for a reliable, specific and sensitive method for detecting and quantifying exosomes. Because exosomes derive from different intracellular origins, it would be advantageous to identify a marker that can be used to detect and quantify essentially all exosomes in a sample.

### SUMMARY OF THE INVENTION

In a first aspect, the present invention provides a method for detecting and/or quantifying exosomes in a sample, the method comprising the steps of i) providing a sample that is known to comprise or suspected of comprising exosomes; ii) contacting a binding agent that specifically binds to the membrane of extracellular vesicles with the sample; and iii) detecting and/or quantifying exosomes by flow cytometry and/or super-resolution microscopy, optionally isolating exosomes. The binding agent comprises a label and a phosphatidylethanolamine-binding lantibiotic. Said label is preferably selected from the group consisting of an enzyme label, a peptide label, a fluorochrome, a ligand, a magnetic or paramagnetic label, and a quantum dot. Said lantibiotic is preferably selected from the group consisting of duramycin and cinnamycin. According to a particularly preferred embodiment, the lantibiotic is cinnamycin.

According to one embodiment, the binding agent is bound by a further binding agent specifically binding to the binding agent or to its label.

According to another embodiment, the binding agent is bound to a solid surface.

According to one embodiment, the sample is a cell culture supernatant, a cell preparation obtained by lysing and/or centrifuging the cells, or a body fluid. The body fluid is preferably selected from the group consisting of plasma, serum, ascites, cerebral fluid, bone marrow, urine, faeces and bronco-alveolar washing.

According to yet another embodiment, the method further comprises prior to step i) one or more of the following steps a) suspending and/or solubilizing the sample; b) ultrafiltration and/or centrifuging the sample; and/or c) enriching the exosome concentration in the sample by size- and/or density-centrifugation.

According to one embodiment, the sample is obtained from a subject known or suspected to suffer from a disease.

According to another embodiment, the method further comprises comparing the quantity of exosomes in the sample of the subject known or suspected to suffer from a disease with the quantity of exosomes known to be present in a sample of a healthy subject, wherein an increase in the quantity of exosomes in the sample of the subject known or suspected to suffer from a disease is indicative of the presence or stage of the disease. Preferably, an increase in the quantity of exosomes in the sample of the subject known or suspected to suffer from a disease is indicative of the presence or stage of cancer.

According to one embodiment, comparing the quantity of exosomes comprises applying CD mapping and t-SNE analysis, NTA tracking, and/or Zetasizer and applying counting beads with flow cytometry.

According to another embodiment, the method further comprises analyzing surface markers and/or the content of the exosomes. Preferably, the surface markers and/or the content of the exosomes comprise one or more of peptides, proteins, microRNA, DNA, and/or RNA. More preferably, analyzing the content of the exosomes comprises DNA mutation analysis, RNA expression, DNA methylation quantification and/or protein expression.

According to a further aspect, the present invention provides a method for diagnosing cancer, said method comprising the steps of a) detecting and isolating exosomes from a sample by a method of the present invention, and b) detecting within the isolated set of exosomes obtained in step a) those exosomes that present cancer antigens.

According to a further aspect, the present invention provides a method for detecting or diagnosing a virus disease, said method comprising the steps of a) detecting and isolating exosomes from a sample by a method of the present invention, and b) detecting within the isolated set of exosomes obtained in step a) those exosomes that present a viral antigen.

According to a preferred embodiment, the viral antigen is a virus surface protein, more preferably a spike protein, most preferably a spike protein of the SARS-CoV-2 or SARS-CoV-1 virus or of any other virus such as Respiratory Syncytial Virus (RSV), Cytomegalovirus (CMV), Hepatitis B and D virus, Influenza virus, Dengue virus, West Nile Virus, Epstein-Barr virus.

According to a further aspect, the present invention provides a method for quantifying and/or qualifying tumor-related exosomes in a sample, said method comprising the steps of a) detecting and isolating exosomes from a sample by a method of the present invention; and b) detecting within the isolated set of exosomes obtained in step a) those exosomes that are tumor-related by using at least one binding agent specifically binding to a tumor antigen.

According to a yet another aspect, the present invention provides a method for monitoring tumor growth, said method comprising the step of periodically quantifying the number of tumor related exosomes in a sample with the method of the present invention, wherein an increase in the number of tumor related exosomes between two quantifications indicates tumor growth.

According to a yet another aspect, the present invention provides a method for monitoring a virus disease, said method comprising the step of periodically quantifying the number of virus-related exosomes in a sample with the method of the present invention.

According to a further aspect, the present invention provides a kit for performing the method of the present invention, the kit comprising (i) a binding agent comprising a label and a phosphatidylethanolamine-binding lantibiotic, wherein the label is preferably selected from the group consisting of an enzyme label, a peptide label, a fluorochrome, a radioactive label, a ligand, a magnetic label, or a quantum dot, and wherein the lantibiotic is preferably selected from the group consisting of duramycin and cinnamycin; and (ii) instructions for binding of said binding agent to exosomes in a sample and for applying flow cytometry and/or super resolution microscopy for detecting, quantifying, enriching or producing exosomes.

Further aspects and embodiments of the present invention are derivable from the following detailed description and the examples.

### FIGURES

FIG. 1: Staining of extracellular vesicles derived from MCF-7 cells with FITC-labeled duramycin. Extracellular vesicles in cell culture supernatant were stained with FITC-labeled duramycin and were analyzed using flow cytometry. Duramycin-positive events are detectable between 10² and 10⁴ representing a mixed population of extracellular vesicles (upper dot plot). As control (lower dot plot), cell culture supernatant was pre-incubated with 1% Triton to solubilize all extracellular vesicles. Staining with FITC-labeled duramycin resulted in no positive events between 10² and 10⁴ confirming the accuracy of the detected signal.

FIG. 2: Signal titration of FITC-labeled duramycin in MCF-7 cell culture supernatant. Extracellular vesicles derived from MCF-7 cell culture supernatant were stained with different concentrations of FITC-labeled duramycin and analyzed by flow cytometry. A concentration-dependent signal decrease in cell culture supernatant stained with FITC-labeled duramycin is shown (dot plots upper row), while no signal is detectable in MCF-7 cell culture supernatant pre-incubated with 1% Triton (dot plots lower row).

FIG. 3: Double staining of MCF-7 derived EVs. Extracellular vesicles generated from MCF-7 cell culture supernatant were stained with FITC-labeled duramycin as well as with an antibody detecting the tetraspanin CD9 located on the surface of EVs. Major part of duramycin-positive EVs is also positive for CD9 (upper dot plot). Pre-incubation of MCF-7 supernatant with 1% Triton results in no duramycin-single positive events or CD9-duramycin double positive events. Analysis of CD9 shows some positive events, an artefact likely caused by antibody aggregation (lower dot plot).

FIG. 4: Double staining of EVs in SKBR 3 cell culture supernatant. EVs derived from SKBR-3 cell culture supernatant were stained for the human epithelial growth factor receptor 2 (ERBB2 or Her2neu) and FITC-labeled duramycin. HER2neu is overexpressed on the surface of tumor cells and is used as diagnostic tool for breast cancer categorization. All Her2neu positive EVs are also positive with FITC-duramycin, while some duramycin-positive events are not positive for Her2neu (dot plot on the left, upper row). Pre-incubation of SKBR-3 cell culture supernatant with 1% Triton results in no positive events for FITC-duramycin and some false positive events for Her2neu which likely represents antibody aggregation (dot plot on the right; upper row). Incubation of DMEM with both agents results in no signal confirming the specificity (dot plot left; lower row). Pre-incubation of DMEM with 1% Triton results in no signal for FITC-duramycin while some positive events are detectable for Her2neu antibody (dot plot right; lower row).

FIG. 5: Double staining of Spike1⁺ EVs in cell culture supernatant. EVs derived from HEKT293 cells transfected with the spike protein of the SARS-CoV-2 virus were stained with FITC-labeled duramycin and the 35B12 antibody which detects the Spike-1 protein on the surface of the EVs. Single stain of Spike1⁺ EVs confirms functionality of 35B12 antibody and staining with FITC-duramycin confirms the detection of EVs in Spike1⁺ supernatant (dot plots left). Pre-incubation of Spike-1⁺ cell culture supernatant with 1% Triton results in no signal for FITC-labeled duramycin, while a clear signal reduction for Spike-1 is visible (dot plots middle). Staining of cell culture supernatant derived from HEKT 293 cells (HEKSN) results in a positive signal for FITC-duramycin which detects EVs independent of their cargi, e.g. spike protein, while no signal is detectable with the 35B12 antibody (dot plots right).

FIG. 6: Enrichment of EVs using ultracentrifugation. MCF-7 cell culture supernatant was centrifuged twice for 15 min. at 1200×g and 20° C., supernatant was centrifuged for 35 min. at 14,000×g and 4° C. The MV pellet were resuspended in PBS and subjected to flow cytometric analysis (MV, microvesicle fraction). The supernatant was ultracentrifuged at 100,000×g and 10° C. for 90 min. The pellet was resuspended in PBS and subjected to flow cytometric analysis (Exo, UZ; exosome fraction). Both samples were stained with FITC-labeled duramycin and signal intensity was compared with flow cytometry. While signal intensity was low when samples were centrifuged twice (dot plot on the left), an additional ultracentrifugation step increased the FITC-duramycin signal intensity significantly (dot plot on the right).

FIG. 7: Exosome detection using CD9 signals after sorting of exosomes. Sorted fractions were analyzed by flow cytometry.

FIG. 8: Size distribution of sorted vesicles. A: Duramycin-FITC⁺ vesicles after sorting using nanoparticle tracking analysis (NTA). B: FITC⁺PE⁺ vesicles after sorting using NTA.

FIG. 9: Structure of duramycin/cinnamycin according to Hullin-Matsuda 2016.

FIG. 10: ELAEXIA labelling of exosomes from plasma and counterstaining with ApoE at fasted state and 1 h, 3 h and 4 h after food intake.

FIG. 11: ELAEXIA labelling of exosomes from plasma and ApoE staining. By means of flow cytometry.

FIG. 12: Flow Cytometry results of staining MCF7 cell culture supernatant with FITC-labelled cinnamycin (top panels) and subsequent staining with anti-CD9 antibody (lower panels).

### DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)” (Leuenberger 1995).

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

The practice of the present invention will employ, unless otherwise indicated, conventional methods of biochemistry, cell biology, and immunology techniques which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

In the following, some definitions of terms frequently used in this specification are provided. These terms will, in each instance of its use, in the remainder of the specification have the respectively defined meaning and preferred meanings.

The term “subject” as used herein refers to an individual, such as a human, a non-human primate (e.g. chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. In a particular meaning, the subject is a mammal. In a preferred meaning, the subject is a human. The subject can be a healthy subject or a subject suffering from or suspected of having one or more diseases. A subject suffering from or suspected of having one or more diseases is also referred to as a patient.

The term “sample” as used herein refers to biological material obtained from a subject. A sample can be obtained from any suitable tissue or biological fluid such as nipple aspirate, blood, serum, plasma, ascites, cerebral fluid, bone marrow, urine, faeces or bronco-alveolar washing. A sample can also be a cell culture supernatant or a cell preparation obtained by lysing and/or centrifuging cells. According to a preferred embodiment, the sample is a body fluid, preferably selected from the group consisting of blood, such as full blood, (blood) plasma, serum, ascites, cerebral fluid, bone marrow, urine, faeces and bronco-alveolar washing. A particularly preferred sample is blood or plasma. Preferably, said sample is provided in a state selected from the group consisting of natural, frozen, lyophilized, preserved, embedded, and all possible combinations thereof. Methods for deriving samples from a subject are well known to those skilled in the art. The sample can be pre-treated by methods known in the art for bringing the sample into a state that allows carrying out the steps of the methods of the present invention. For example, if the sample is in a solid, semi-solid, or essentially solid or semi-solid state, the sample can be suspended and/or solubilized. The sample can be further pre-treated by removing e.g. cellular debris and larger cellular components. Such methods include but are not limited to centrifugation techniques, particularly ultrafiltration. According to a preferred embodiment, the sample is centrifuged at around 10,000 to 15,000 g, preferably about 12,000 g for separating larger solid particles such as cellular debris and maintaining EVs in the supernatant. The sample after pre-treatment and before applying the methods of the present invention preferably does not comprise microvesicles larger than 1,000 nm in diameter, preferably not larger than 800 nm, 700 nm, 600 nm, 500 nm, 400 nm, 300 nm, 200 nm, 150 nm in diameter. Most preferably, the sample after pre-treatment and before applying the methods of the present invention does not comprise microvesicles having a diameter of more than 100 nm. It is to be understood that the diameter of such microvesicles refers to the average diameter of a plurality or a population of such microvesicles. The sample can be further pre-treated by enriching the exosome concentration in the sample. Suitable methods include size- and/or density-centrifugation. These methods are well known to the person of ordinary skill in the art. However, such enriching in exosomes is not necessary and the methods of the invention can be performed directly on the sample. Thus, according to a preferred embodiment of the invention, the method does not comprise any purification step of the exosomes contained in the sample in advance to the claimed method, i.e. no pre-purification, such as by ultracentrifugation, density centrifugation, fractionation and/or the use of markers for micro-vesicles or exosomes (e.g. Rab4 and/or Rab11), markers for tumors, or viral markers, that may bind to all or a subfraction of the exosomes contained in said sample. The method of the invention preferably also does not require any previous exosome enrichment using agents that bind to an exosome or to an exosome sub-population. According to one embodiment, the exclusion of a pre-purification step includes an exclusion of cellular lysis and/or removing of cellular debris in the sample. According to an alternative embodiment, the exclusion of a pre-purification step does not include an exclusion of cellular lysis and/or removing of cellular debris in the sample. In accordance with the present invention, the method can thus be directly performed on a respective sample, such as on plasma.

The present inventors developed an innovative procedure that enables to analyze single EVs and specifically exosomes isolated from samples such as human blood plasma. During the course of experiments leading to the present invention, the inventors found that current markers for EVs such as CD9, CD63 and CD81 do not detect the entire population of EVs in the blood of humans. Using CD9, CD63 and CD81 as marker also led to detection of EVs derived from platelets. Other markers such as Calcein or CFSE, which penetrate into exosomes and get activated through esterases, also do not detect the entire EV population because these activations are ATP dependent and this might explain the heterogeneity in staining of exosomes.

It is known that exosome biogenesis occurs in cells and at the molecular level inside the Multi-Vesicular Body (MVB) compartment. Intralumenal vesicles, precursors of exosomes, are generated inside the MVB compartment (late endosomes). The budding vesicle membrane is based on the exosome lipid composition and the absence of phospholipid transmembrane asymmetry. The donor membrane for the budding vesicle is the MVB membrane (Subra 2007). Lipids such as phosphatidylserine and phosphatidylethanolamine, usually in the inner leaflet of the membrane of cells, form through this inward budding of MVBs the outer leaflet of the exosome membrane. It is, however, also known that phosphatidylethanolamine (PE)-binding duramycin and cinnamycin exhibit cytotoxicity, and that they can even cause membrane distortion and induce PE translocation at micromolar concentration (Makino 2003).

The present inventors found that despite the prejudice in the art, PE-binding lantibiotics such as duramycin and cinnamycin may nevertheless be used for reliably detecting and quantifying essentially all exosomes in a sample and not just a fraction or subpopulation thereof. Therefore, according to a preferred embodiment, the method of the present invention comprises the steps of i) providing a sample that is known to comprise or suspected of comprising exosomes; ii) contacting a binding agent that specifically binds to the membrane of extracellular vesicles with the sample; and iii) detecting and/or quantifying exosomes by flow cytometry and/or super-resolution microscopy, and optionally isolating exosomes. According to the present invention, the binding agent comprises a label and a PE-binding lantibiotic. The PE-binding lantibiotic is preferably selected from the group consisting of duramycin and cinnamycin.

Cinnamycin possesses an arginine at amino acid position 2, and not a lysine. The amino group in the amino acid arginine becomes—if embedded in a peptide—a guanidino group and, therefore, this amino group is not a free amino group available for labelling. Therefore, cinnamycin can only be labelled via an amino group at the N-terminus, whereas duramycin has two amino groups for labelling, one at amino acid position 2 and one at the N-terminus. Thus, in contrast to duramycin, cinnamycin can only be labelled at one position. Therefore, according to a preferred embodiment of the present invention, the lantibiotic is cinnamycin.

The method of the invention allows isolating exosomes without isolating any other microvesicles in any substantial amounts. Thus, according to one embodiment, the method of the invention isolates essentially no microvesicles other than exosomes. According to a preferred embodiment, the method of the invention isolates no microvesicles other than exosomes.

According to a preferred embodiment, the exosomes are enriched in the sample based e.g. on size and/or density before the sample is contacted with the PE-binding agent. Such enrichment of exosomes can be performed e.g. by density centrifugation or ultra-centrifugation using e.g. a Pancoll gradient (PAN-Biotech GmbH). Other suitable gradients include but are not limited to Ficoll and Ficoll-Paque (both GE Healthcare), and Biocoll (Biochrom GmbH). Methods of performing ultra-centrifugation are well known to the person of skill in the art and are disclosed for example in Li et al., 2017, which is herein incorporated by reference.

The term “agent” as used herein denotes a chemical compound, a mixture of chemical compounds, a biological macromolecule, or an extract made from biological materials.

The term “binding agent” as used herein pertains to any agent capable of specifically and/or selectively binding to a specific biological structure. In the context of the present invention, the term “phosphatidylethanolamine-binding” is used to denote agents that specifically and/or selectively bind to phosphatidylethanolamine (PE), in particular to PE present in the membrane of exosomes.

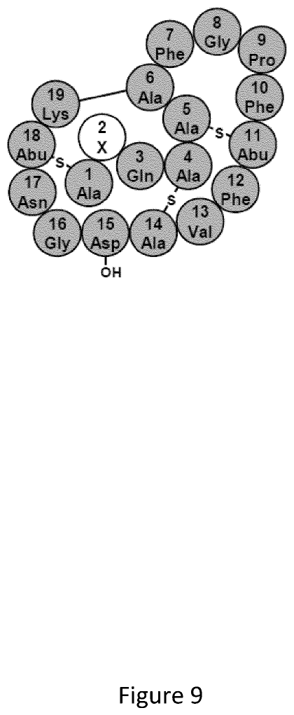

According to the present invention, the PE-binding agent is a PE-binding lantibiotic. Lantibiotics are known as a class of polycyclic peptide antibiotics that contain the characteristic thioether amino acids lanthionine or methyllanthionine, as well as the unsaturated amino acids dehydroalanine, and 2-aminoisobutyric acid. According to a preferred embodiment, the PE-binding lantibiotic is duramycin or cinnamycin having a sequence at least 60% identical to SEQ ID NO: 1 (duramycin) or SEQ ID NO: 2 (cinnamycin), or at least 65%, 70%, 75%, 80%, 85%, 90%, 95% or 100% identical to SEQ ID NO: 1 or 2, and being capable of specifically and/or selectively binding PE. Duramycin and cinnamycin are 19-amino acid long peptides that bind the head group of PE with high specificity and affinity, as described in e.g. Navarro 1985 and in Zhao 2008. According to a preferred embodiment, the PE-binding lantibiotic is duramycin. According to an alternatively preferred embodiment, the PE-binding lantibiotic is cinnamycin. Cinnamycin and duramycin are both tetracyclic peptides differing only by a single amino acid in position 2: arginine for cinnamycin and lysine for duramycin. They have a high degree of sequence and structural homology. They are produced by Streptomyces species and Streptoverticillium cinnamoneus, respectively. The structure of duramycin and cinnamycin according to Hullin-Matsuda et al., Biochimie (2016) doi: 10.1016/j.biochi.2016.09.020 is shown in FIG. 9. The sequence of duramycin and of cinnamycin is as follows:

(duramycin)
SEQ ID NO: 1
AKQAAAFGPFXFVADGNXL
and
(cinnamycin)
SEQ ID NO: 2
ARQAAAFGPFXFVADGNXL,

wherein X denotes α-aminobutyric acid.

According to the present invention, the PE-binding lantibiotic comprises a label. As used herein, the term “label” or “labeled” refers to incorporation of a detectable marker, e.g. by incorporation of a respectively labeled amino acid or by attachment of a respective marker to the PE-binding lantibiotic. The term “label” as used herein preferably encompasses a detectable moiety that is either directly or indirectly attached to the PE-binding lantibiotic. Such a direct or indirect attachment can be by one or more covalent or non-covalent bonds. If the label is indirectly attached to the PE-binding lantibiotic, one or more linker preferably links the label to the PE-binding lantibiotic. The linker can be any type of linker that allows binding of the PE-binding lantibiotic to PE and at the same time allows detection or binding of the label by a further binding agent that specifically binds the label. The linker is preferably a polypeptide linker. The label to be used in the context of the present invention can be selected from the group consisting of but not limited to an enzymatic label, a peptide label, a fluorochrome label, a ligand label, a magnetic or paramagnetic label, and a quantum dot. A ligand label can be, for example, biotin, which can then be bound by avidin or streptavidin. It is to be understood that alternatively streptavidin or avidin can be used as label, which is then bound by biotin. It is further to be understood that a specific label may fall under more than one of the terms used herein for identifying different groups of labels. For example, a ligand label can be at the same time a peptide label. A non-limiting example of such a label is the preferred label Strep-tag, which falls under the group of ligand labels and peptide labels. Examples of ligand and peptide labels include but are not limited to biotin and avidin/streptavidin and other labels such as His-tag, FLAG-tag and Strep-tag. According to a preferred embodiment, the label is a Strep-tag label, preferably Strep-tag having the amino sequence of WSHPQFEK (SEQ ID NO: 3) or Twin-StrepTag (IBA Lifesciences, Gottingen) having the sequence WSHPQFEK-GGGSGGGSGG-SA-WSHPQFEK (SEQ ID NO: 4). Strep-tags are known to bind to streptavidin, Streptactin and Steptactin XT with high affinity.

According to a particularly preferred embodiment, the label is a fluorochrome (also termed fluorophore). The term “fluorochrome” and likewise “fluorophore” as used herein refers to a non-protein fluorescent chemical compound that can re-emit light upon light excitation. The fluorochrome is preferably selected from the group consisting of but not limited to xanthene derivatives such as fluorescein and its derivatives, rhodamine, Oregon green, eosin, and Texas red; cyanine derivatives such as cyanine, indocarbocyanine, oxacarbocyanine, thiacarbocyanine, and merocyanine; squaraine derivatives and ring-substituted squaraines, including Seta and Square dyes; squaraine rotaxane derivatives such as See Tau dyes; naphthalene derivatives; coumarin derivatives; oxadiazole derivatives such as pyridyloxazole, nitrobenzoxadiazole and benzoxadiazole; anthracene derivatives such as anthraquinones, including DRAQ5, DRAQ7 and CyTRAK Orange; pyrene derivatives such as cascade blue; oxazine derivatives such as Nile red, Nile blue, cresyl violet, oxazine 170; acridine derivatives such as proflavin, acridine orange, and acridine yellow; arylmethine derivatives such as auramine, crystal violet, and malachite green; tetrapyrrole derivatives such as porphin, phthalocyanine, and bilirubin; and dipyrromethene derivatives such as BODIPY, and aza-BODIPY. Preferred fluorochromes have a size of between 200 and 1,000 Dalton. A preferred fluorochrome is Atto 488. A further particularly preferred fluorochrome according to the present invention is fluorescein isothiocyanate (FITC).

According to one particularly preferred embodiment, the PE-binding lantibiotic is duramycin or cinnamycin, and the label is attached to the first or second amino acid position of duramycin (i.e. at position Ala1 or Lys2 with respect to SEQ ID NO: 1) or the label is attached to the first or second amino acid position of cinnamycin (i.e. at position Ala1 with respect to SEQ ID NO: 2). A preferred fluorochrome is Atto 488 or FITC, most preferred FITC. Particularly preferred embodiments are duramycin labeled with FITC at position Ala1 or Lys2, and cinnamycin labeled with FITC at position Ala1. A preferred binding agent in the context of the present invention is duramycin labeled with FITC, preferably at position Ala1 or Lys2. An alternatively preferred binding agent in the context of the present invention is cinnamycin labeled at position Ala1, preferably with FITC. According to a particularly preferred embodiment, the binding agent is cinnamycin labeled at position Ala1 with FITC. According to an alternative preferred embodiment, the binding agent is cinnamycin labeled at position Ala1 with Strep-tag. As set forth above, a linker (preferably a peptide linker) can be present between the PE-binding lantibiotic and the label, such as between the cinnamycin and the Strep-tag.

According to the present invention, the exosomes are detected and/or quantified by flow cytometry and/or super-resolution microscopy. The term “flow cytometry” as used herein refers to technique used to detect and measure physical and chemical characteristics of a population of cells or particles. Flow cytometry is a sorting technique that rapidly analyzes single cells but also other particles as they flow past single or multiple detectors while suspended in solution. Cell sorting in general describes the process of purifying or enriching cell populations based on the presence or absence of specific physical characteristics. Using flow cytometry, the exosomes in the sample that are marked by the PE-binding agent will be identified based on the respective label and will be separated from other components in the sample. Flow cytometry is an established method in the art and the person of ordinary skill will readily identify various possible flow cytometers and respective methods for putting the invention into practice. Flow cytometers that can be used in this context are commercially available and well established in the field of cell biology. A preferred flow cytometry technique is fluorescence flow cytometry such as fluorescence-activated cell sorting (FACS) which requires a fluorescent label on the PE binding agent such as a fluorochrome or quantum dots. Any suitable flow cytometer or cell sorter can be used for putting the invention into practice such as but not limited to spectral cell analyzers or sorters. In flow cytometers with sorting capabilities, the instrument detects cells using parameters such as cell size, morphology, and protein expression. Droplet technology is then used to sort cells and recover the subsets. This principle can also be applied for purifying or enriching e.g. cell components such as exosomes. Other detection methods might be luminometer, fluorometer, confocal laser microscopy, or super-resolution microscopy analysis and quantification.

For detecting the binding agent, according to one embodiment of the present invention, said binding agent can be bound by a further (second) binding agent specifically binding to the binding agent that binds PE (first binding agent). The binding of the further second binding agent to the first binding agent can be at any part of the first binding agent such as the PE binding lantibiotic or to the label of said first binding agent. The further (second) binding agent can be, for example, an antigen binding agent such as an antibody or an antigen binding fragment of an antibody. The second binding agent may also comprise a label which can be selected from the group consisting of an enzymatic label, a fluorochrome, a magnetic label such as magnetic beads, and a peptide label as disclosed herein. Examples of peptide labels include but are not limited to biotin and avidin/streptavidin and other labels such as His-tag, FLAG-tag and Strep-tag. According to a preferred embodiment, the label is a Strep-tag label, preferably Strep-tag having the amino sequence of WSHPQFEK (SEQ ID NO: 3) or Twin-StrepTag (IBA Lifesciences, Gottingen) having the sequence WSHPQFEK-GGGSGGGSGG-SA-WSHPQFEK (SEQ ID NO: 4). Strep-tags are known to bind to streptavidin, Streptactin and Steptactin XT with high affinity. Commonly used enzymatic labels, fluorochromes, and magnetic labels can be used in the context of the present invention without any specific limitation thereto. Respective examples include but are not limited to FITC, rhodamine, lanthanide phosphors for fluorescent labels; horseradish peroxidase, β-galactosidase, luciferase, and alkaline phosphatase for enzymatic labels; and chemiluminescent labels. Quantum dots can also be used as label. Quantum dots are semiconductor nanocrystals that have broad excitation spectra, narrow emission spectra, tunable emission peaks, long fluorescence lifetimes, negligible photobleaching, and ability to be conjugated to proteins. There is also no specific limitation regarding the use of quantum dots in the present invention.

According to a preferred embodiment, the label is selected from the group consisting of a fluorochrome, a ligand such as biotin or a StrepTag peptide, a magnetic or paramagnetic label, and a quantum dot. According to one specific embodiment, the label is not an enzyme label or an enzymatic label, such as e.g. horseradish peroxidase. According to a further specific embodiment, the label is not a gold label.

In certain embodiments, labels are attached by spacer arms of various lengths to reduce potential steric hindrance. Such spacers can be, for example, chemical spacers or amino acid spacers.

If the further (second) binding agent is an antigen binding agent, it “specifically binds” a target antigen when the dissociation constant (K_{d}) is ≤10⁻⁷ M. The binding agent specifically binds its antigen with “high affinity” when the K_{d} is ≤5×10⁻⁹ M, and with “very high affinity” when the K_{d} is ≤5×10⁻¹⁰ M, preferably when the K_{d} is ≤5×10⁻¹¹ M.

If the binding agent is an antigen binding agent, it is “selective” when it binds to one target more tightly than it binds to a second target.

In an alternative embodiment of the present invention, the first (PE-) binding agent and/or the second binding agent is covalently or non-covalently bound on a solid surface, as in an array setting. Such array construct may be used for binding the exosomes to respective probes comprising the binding agents. Accordingly, the present invention also provides an array comprising the first and/or second binding agents, and the use of such array for detecting and/or quantifying exosomes in a sample. If bound to an array, the exosomes can be released after washing the array to remove any contaminants.

The conditions allowing binding of the first and/or the second binding agent to the sample depend in particular on the type of binding agent used and on the type of sample. The conditions can be easily determined by a person of ordinary skill in the art.

Detecting the first and/or second binding agent can be performed by any method known in the art. If a label is attached, it is envisioned to detect the label. For example, biotin moieties can be detected by marked avidin (e.g. streptavidin containing a fluorescent marker or enzymatic activity that can be detected by optical or colorimetric methods), and fluorescent labels can be detected by exciting the fluorophore and detecting the emitted fluorescence of the fluorophore. Respective detection methods are well known to the person of skill in the art.

According to the present invention, the exosomes may also be detected and/or quantified by applying super-resolution microscopy (Neice 2010 and Stockert 2017; both incorporated herein by reference). While conventional microscopy techniques such as electron microscopy have the capability to resolve individual EVs, they do not easily allow detection of different markers and are limited to fixed cells. With conventional light microscopy techniques, such as confocal light microscopy, a number of proteins can be labeled but the small size of EVs means that the majority fall well below the resolution limit of light microscopy, restricting the usefulness of these techniques in identifying different sub-populations of vesicles. Super-resolution microscopy on the other hand enables detection and quantification at the sub-vesicular level. This technique enables single-molecule fluorescence microscopy and can be used to follow respectively labeled vesicles in solution. Based on the vesicles' trajectories and corresponding diffusion measurements, one can quickly estimate the size distribution and concentration of an EV population in a respective sample. Super-resolution microscopy for detecting extracellular vesicles is described e.g. in Gebara 2022, incorporated herein by reference.

The methods of the present invention are preferably carried out with samples obtained from a subject suffering from or suspected of having one or more diseases. In such cases, the method may further comprise the step of comparing the quantity of exosomes in the sample of the subject known or suspected to suffer from a disease with the quantity of exosomes known to be present in a sample of a healthy subject. An increase in the quantity of exosomes in the sample of the subject known or suspected to suffer from a disease is then indicative of the presence or stage of the disease. According to a preferred embodiment, an increase in the quantity of exosomes in the sample of the subject known or suspected to suffer from a disease is indicative of the presence or stage of a virus infection or cancer.

According to the present invention, when comparing the quantity of exosomes, this step is preferably performed by applying CD mapping, t-SNE analysis, nanoparticle tracking analysis (NTA), or using a Zetasizer and applying counting beads with flow cytometry. CD mapping includes identifying expression of different Cluster of Differentiation (CD) proteins on the enriched or isolated exosomes and comparing the CD protein expression of one sample with the CD protein expression of another sample. The CD expression profile can then be combined in form of a map. T-distributed Stochastic Neighbor Embedding (t-SNE) is a machine learning algorithm for visualization. It is a nonlinear dimensionality reduction technique well-suited for embedding high-dimensional data for visualization in a low-dimensional space of two or three dimensions. Specifically, it models each high-dimensional object by a two- or three-dimensional point in such a way that similar objects are modeled by nearby points and dissimilar objects are modeled by distant points with high probability. The technique of t-SNE is well known to the person of skill in the art.

According to a preferred embodiment, the method further comprising analyzing surface markers and/or the content of the exosomes. Surface markers are particularly presented on the surface of an exosome. Exosomes “presenting” or “carrying” a molecule such as a peptide, protein or antigen as referred to herein means that the molecule is at least partially accessible from the outside of the exosome. For example, a respective molecule may be located on the exosome's outer membrane or at least a portion of said molecule may be located on the exosome's outer membrane. A respectively presented or carried molecule may also spun or extend through the exosome's membrane.

The term “extra-vesicular part” as used herein denotes the part of a molecule such as a peptide or protein present in or on an exosome that is accessible from the outside of the exosome.

An analysis of the surface markers and/or the content of the exosomes allows a further characterization of the exosomes and drawing conclusions on e.g. the health status of the subject from which the sample is derived. According to a particularly preferred embodiment, said surface markers and/or said content of the exosomes comprises one or more of peptides, proteins, microRNA, DNA, and/or RNA. The content of the exosomes can be analyzed by applying e.g. DNA mutation analysis, analyzing RNA expression, quantifying DNA methylation and/or quantifying/determining protein expression.

In order to develop, for example, a tumor-specific exosome test, it is necessary to detect and purify all or at least the majority of exosomes derived from normal and tumor cells, and to isolate and enrich tumor-specific exosomes (oncosomes) with high purity and quality for subsequent tumor biomarker analysis in clinical diagnostics. By using for example the blood from cancer patients as test sample, exosome characterization may serve as “liquid biopsy”, allowing an alternative, less invading sampling, which can even be applied if tumor tissue is not directly accessible. A respective analytical method further enables to screen for cancer, to monitor therapy, disease progression and recurrence. Accordingly, the present invention provides a method for diagnosing cancer. The method comprises the steps of a) detecting and isolating exosomes from a sample by the method of the present invention, and b) detecting within the isolated set of exosomes obtained in step a) those exosomes that present cancer antigens. The present invention is not particularly limited to detecting any specific cancer antigen and the person of ordinary skill in the art will select one or more suitable cancer antigens to be detected. A preferred cancer antigen is GPER-1 (G protein-coupled estrogen receptor 1). A further preferred cancer antigen is HER2. A significantly increased number of exosomes presenting the cancer antigen and preferably GPER-1 or HER2 compared with a reference sample of a healthy person is indicative of the person being at risk or suffering from cancer. Preferably, GPER-1 or HER2 is detected by using a binding agent specifically and/or selectively binds GPER-1 and HER2, respectively. Preferably, said binding agent is an antigen-binding agent, and more preferably an antibody against GPER-1 or HER2. The method of diagnosing cancer can be preferably used for diagnosing breast cancer. Alternatively or in addition to detecting GPER-1 or HER2, also GPER-5, CD247 (T cell surface glycoprotein CD3 zeta chain; Cluster of Differentiation 247) and/or phophatidylserine can be used for detecting such oncosomes. Further exosome surface markers associated with cancer that can be analyzed according to the present invention are CD49b, CD90, and CD202b.

According to a further aspect, the present invention provides a method for quantifying and/or qualifying tumor-related exosomes in a sample. The method comprises the steps of a) detecting and isolating exosomes from a sample by the method of the present invention, and b) detecting within the isolated set of exosomes obtained in step a) those exosomes that are tumor-related by using at least one binding agent specifically binding to a tumor antigen.

Such tumor related exosomes (or oncosomes) can be identified by comparing the exosome population of a healthy person with the exosome population of a person suspected of having cancer or already known to suffer from cancer. The tumor antigen can be selected from any tumor antigen known in the art. According to a preferred embodiment, the tumor antigen is selected from the group consisting of GPER-1, GPER-5, HER2, CD247, and phophatidylserine. Further exosome surface markers associated with cancer that can be analyzed according to the present invention are CD49b, CD90, CD274 and CD202b.

According to a further aspect, the present invention provides a method for monitoring tumor growth. The method comprising the steps of periodically quantifying the number of tumor related exosomes in a sample with the method of the present invention. An increase in the number of tumor related exosomes between two quantifications is then indicative of tumor growth.

According to a further aspect, the present invention provides a method for diagnosing a virus disease. The method comprises the steps of a) detecting and isolating exosomes from a sample by the method of the present invention, and b) detecting within the isolated set of exosomes obtained in step a) those exosomes that present a viral antigen. According to a preferred embodiment, the viral antigen is a virus surface protein. The virus surface protein is preferably a spike protein, more preferably a spike protein of the SARS-CoV-2 or SARS-CoV-1 virus. The virus antigen can be also a subunit of the spike protein e.g. subunit 1 or 2, or peptides derived thereof. Alternatively, the virus protein can be associated with any other virus infection such as for example Hepatitis B, C, D, or E, RSV, EBV, influenza A/B, West-Nile-, Zika-, Dengue- or Ebola-virus infections. According to a particularly preferred embodiment, the viral antigen is a spike protein. Particularly preferred spike proteins are those of the SARS-CoV-2 or SARS-CoV-1 virus. Alternatively, the spike protein can be from any other virus.

The present invention further provides a method for monitoring a virus disease. The method comprises the step of periodically quantifying the number of virus-related exosomes in a sample with the method of the present invention. The presence of virus-related exosomes containing virus derived proteins allows diagnosing and monitoring of the virus disease. The virus derived protein preferably is a virus antigen. According to an embodiment, the virus antigen is a virus surface protein. The virus surface protein is preferably a spike protein, more preferably a spike protein of Riboviria, most preferably of the SARS-CoV-2 or Sars-CoV-1 virus. The virus antigen can be also a subunit of the spike protein e.g. subunit 1 or 2, or peptides derived thereof. Alternatively, the virus protein can be associated with any other virus infection such as for example Hepatitis B, C, D, or E, RSV, EBV, influenza A/B, West-Nile-, Zika-, Dengue- or Ebola-virus infections.

Any of the methods described herein may further comprise the step of comparing the quantity of exosomes, preferably of disease related exosomes, in the sample of the subject known or suspected to suffer from a disease with the quantity of similar exosomes known to be present in a sample of a healthy subject. An increase in the quantity of the exosomes in the sample of the subject known or suspected to suffer from a disease is then indicative of the presence or stage of the disease. The term “disease related exosomes” is intended to refer to exosomes containing peptides associated with a disease such as viral peptides and antigens as well as cancer peptides and antigens, e.g. as described herein. A disease to be detected or monitored according to the invention is preferably cancer. In such methods, comparing the quantity of exosomes may comprise applying CD mapping and t-SNE analysis, NTA Tracking, and using a Zetasizer by e.g. applying counting beads with flow cytometry.

Additional analysis of markers such as proteins, peptides and/or antigens in addition to those associated with a disease (such as viral or cancer peptides and/or antigens) may allow localizing the disease to a specific organ or tissue. For example, a disease related exosome may additionally carry or comprise one or more markers for a specific organ or tissue such as cardiac troponin for the heart. Thus, additionally identifying in a population of disease related exosomes organ or tissue specific markers or markers associated with a group of tissues or organs allows associating disease related exosomes to a tissue or an organ, thereby associating the disease to the tissue or organ. Therefore, for further investigating the enriched or isolated exosome populations of a sample, further surface markers and/or the content of the exosomes can be analyzed. Such further surface markers include but are not limited to amyloid-beta, 14-3-3 protein, Actin, ADAM10, Alix, alpha-Enolase, alpha-Synuclein, Aminopeptidase N, Annexin 5A, Annexin A2, AP-1, ATP citrate lyase, ATPase, Basigin, Caveolin-1, Clathrin, Claudin-1, Cofilin-1, EGFR, Ep-CAM, ICAM, HLA-ABC, prostate specific antigen, Rab-14, Rab-7, Syndecan, Tumor-Associated Glycoprotein, Tetraspanin-8, Tsg101, vacuolar-sorting protein 35, CD2, CD3, CD5, CD8, CD9, CD11a, CD11b, CD11c, CD13, CD29, CD37, CD41, CD44, CD49d, CD49f, CD62L, CD63, CD68, CD80, CD81, CD86, CD90, CD142, CD146, CD163, CD192, and CD202b.

Specific examples of markers that may associate a disease such as a viral infection with a specific organ or tissue include surfactant associated protein A (SP-A) and surfactant associated protein B (SP-B) for the lung, cardiac troponin for the heart, von Willebrand factor and CD31/PECAM-1 for endothelium, Enolase-2 (ENO2) and neuron specific enolase (NSE) for the brain or neuro tissues, Asialoglycoproteinreceptor 1 (ASGR-1) for the liver, and Aquaporin 6 for the kidney. These markers are particularly suitable for associating a virus infection with a specific organ or tissue as origin of virus replication, more preferably for associating a SARS-CoV virus or Influenza A/B virus infection with a specific organ or tissue, and most preferably a SARS-CoV 2 virus infection.

Analyzing the content of the exosome may include lysis of the exosomes. The content to be analyzed include but are not limited to peptides, proteins, microRNA, DNA, and/or RNA such as mRNA. Proteins to be analyzed typically include but are not limited to platelet derived growth factor receptor, lactadherin, transmembrane proteins and lysosome associated membrane protein-2B, membrane transport and fusion proteins like annexins, flotillins, GTPases, heat shock proteins, tetraspanins, proteins involved in multivesicular body biogenesis, as well as lipid-related proteins and phospholipases. The analysis of the contents may include DNA mutation analysis, RNA expression, DNA methylation quantification and/or protein expression, as well as fluorescence flow cytometry. In case of analyzing nucleic acids, these can be for example quantified to identify their profiles by methods known in the field and involving for example RT-PCR. Exosomes which are released from virus infected cells contain Rab11 which allows virosome identification and applying a specific second antibody directed against a specific viral protein for diagnosing and monitoring viral diseases. The analysis of the exosome contents and surface proteins and peptides is not limited to detecting and monitoring cancer or viral infections but may be generally used for detecting or characterizing many medical conditions and diseases.

According to a further aspect, the present invention provides a kit for performing any of the methods of the present invention as described herein. The kit comprises a binding agent comprising a label and a phosphatidylethanolamine-binding lantibiotic, and instructions for binding of said binding agent to exosomes in a sample and for applying flow cytometry and/or super resolution microscopy for detecting, quantifying, enriching or producing exosomes. The binding agent is as defined above. The label is preferably selected from the group consisting of an enzyme label, a peptide label, a fluorochrome, a ligand, a magnetic label, or a quantum dot, and the lantibiotic is preferably selected from the group consisting of duramycin and cinnamycin. The kit may further comprise a further (second) binding agent as defined herein above, which second binding agent may comprise a label as defined above. The kit may further comprise means and/or instructions for preparing the sample before the binding agent(s) is/are added to the sample.

The invention is further described by way of the following examples which are to be construed as merely illustrative and not limitative of the scope of the invention.

### ITEMS OF THE INVENTION

The present invention also pertains to the following items:

Item 1: A method for producing an exosome enriched fraction from a sample, the method comprising the steps of: i) providing a sample that is known to comprise or suspected of comprising exosomes;
ii) contacting a binding agent that specifically binds to the membrane of extracellular vesicles with the sample; and
iii) detecting and/or quantifying exosomes by flow cytometry and/or super-resolution microscopy, and optionally isolating exosomes;
wherein the binding agent comprises a label and a phosphatidylethanolamine-binding lantibiotic,
wherein the label is preferably selected from the group consisting of an enzyme label, a peptide label, a fluorochrome, a radioactive label, a ligand, a magnetic or paramagnetic label, and a quantum dot, and
wherein the lantibiotic is preferably selected from the group consisting of duramycin and cinnamycin.

Item 2: The method of item 1, wherein:
(a) the binding agent is bound by a further binding agent specifically binding to the binding agent or to its label; or
(b) the binding agent is bound to a solid surface.

Item 3: The method of item 1 or 2, wherein the sample is a cell culture supernatant, a cell preparation obtained by lysing and/or centrifuging the cells, or a body fluid.

Item 4: The method of item 3, wherein the body fluid is selected from the group consisting of plasma, ascites, cerebral fluid, bone marrow, urine, faeces and bronco-alveolar washing.

Item 5: The method of any one of items 1 to 4, wherein the method further comprises prior to step i) one or more of the following steps:
a) suspending and/or solubilizing the sample;
b) ultrafiltration and/or centrifuging the sample; and/or
c) enriching the exosome concentration in the sample by size- and/or density-centrifugation.

Item 6: The method according to any one of items 1 to 5,
wherein the sample is obtained from a subject known or suspected to suffer from a disease, wherein the method further comprises comparing the quantity of exosomes in the sample of the subject known or suspected to suffer from a disease with the quantity of exosomes known to be present in a sample of a healthy subject, wherein an increase in the quantity of exosomes in the sample of the subject known or suspected to suffer from a disease is indicative of the presence or stage of the disease,
preferably wherein an increase in the quantity of exosomes in the sample of the subject known or suspected to suffer from a disease is indicative of the presence or stage of cancer.

Item 7: The method of item 6, wherein comparing the quantity of exosomes comprises applying CD mapping and t-SNE analysis., NTA Tracking, Zetasizer and applying counting beads with flow cytometry.

Item 8: The method according to any one of items 1 to 7, further comprising analyzing surface markers and/or the content of the exosomes,
preferably wherein the surface markers and/or the content of the exosomes comprise one or more of peptides, proteins, microRNA, DNA, and/or RNA, more preferably wherein analyzing the content of the exosomes comprises DNA mutation analysis, RNA expression, DNA methylation quantification and/or protein expression.

Item 9: A method for diagnosing cancer, comprising:
a) detecting and isolating exosomes from a sample by a method of any one of items 1 to 8, and
b) detecting within the isolated set of exosomes obtained in step a) those exosomes that present cancer antigens.

Item 10: A method for detecting or diagnosing a virus disease, comprising:
a) detecting and isolating exosomes from a sample by a method of any one of items 1 to 8, and
b) detecting within the isolated set of exosomes obtained in step a) those exosomes that present a viral antigen,
preferably wherein the viral antigen is a virus surface protein, more preferably a spike protein, most preferably a spike protein of the SARS-CoV-2 or Sars-CoV-1 virus or of any other virus.

Item 11: A method for quantifying and/or qualifying tumor-related exosomes in a sample, said method comprising the steps of:
a) detecting and isolating exosomes from a sample by a method of any one of items 1 to 8; and
b) detecting within the isolated set of exosomes obtained in step a) those exosomes that are tumor-related by using at least one binding agent specifically binding to a tumor antigen.

Item 12: A method for monitoring tumor growth, said method comprising the step of:
periodically quantifying the number of tumor related exosomes in a sample with the method according to item 11, wherein an increase in the number of tumor related exosomes between two quantifications indicates tumor growth.

Item 13: A method for monitoring a virus disease, said method comprising the step of:
periodically quantifying the number of virus-related exosomes in a sample with the method according to any one of items 1 to 8 or item 10.

Item 14: A kit for performing the method according to any one of items 1 to 13, comprising:
(i) a binding agent comprising a label and a phosphatidylethanolamine-binding lantibiotic,
   wherein the label is preferably selected from the group consisting of an enzyme label, a fluorochrome, a peptide label, a radioactive label, a ligand, a magnetic label, or a quantum dot, and wherein the lantibiotic is preferably selected from the group consisting of duramycin and cinnamycin, more preferably wherein the lantibiotic is duramycin; and
(ii) instructions for binding of said binding agent to exosomes in a sample and for applying flow cytometry and/or super resolution microscopy for detecting, quantifying, enriching or producing exosomes.

### EXAMPLES

### Devices and Reagents

In the examples of the invention, the following devices and reagents have been used:

### FACS Device:

Sony SP6800 Spectral Analyzer

### Pancoll Gradient:

PAN Biotech, Density: 1.077 g/ml

### PEG6000:

Molecular Biology grade, Merck

### Centrifuges:

Heraeus Multifuge X3 R (Cat. no.: 75004515; Serial number: 41615170 Thermo Scientific)
Heraeus Fresco 17 (Cat. no. 75002420; Serial number: 41284997; Thermo Scientific)
Optima LE-80K (Beckman Coulter)

### Antibodies:

CD9 PE (anti-human CD9 antibody labeled with phycoerythrin) (#312106; 20 μg/ml; Biolegend)
Her2neu PE (anti-human CD340 antibody labeled with phycoerythrin) (#324406; 50 μg/ml; Biolegend)
Antibodies with indicated labels against HLA-ABC-PE-Cy5, CD5-FITC, CD8-PE-Cy7, CD13-BV421, CD81-PE-Dazzle 594, CD41-PacBlue, CD68-FITC, CD86-BV650, GPER-1-DyeLight 405, Rab5-PE.

### Further Reagents:

Triton-X100 (PanReac AppliChem)
Counting Beads (Count Bright Absolute Counting Beads, ThermoFisher, Cat. No.: #C36950, Lot.: 2466363)
NaCl (0.9%; sterile solution; Braun)
RPMI (+) L-Glutamine cell culture medium (Ref no: 21875-034; Gibco)
DMEM Medium high glucose (Cat. no.: D5796; 500 ml; Sigma Life Science)

### Flow Cytometer:

SA 3800 (Sony)
Used Settings (exosome settings)

| Equipment: | ID | Configuration: |
|---|---|---|
| Flow Cytometer | Sony SA3800 | Standard |
| Boost | | Normal |
| Sample Flow Rate | | 1.0 |
| Laser Wavelength settings | | 405 nm; 488 nm |
| Windows extension | | normal |
| Threshold CH/Value % | | SSC/0.1% |
| FSC Gain | | 11 |
| SSC Voltage % | | 28 |
| Fluorescent PMT Voltage % | | 65% |

### Example 1

Labeling of Duramycin with FITC:

Duramycin was labeled with FITC using a column-based kit from abcam (EZLabel Protein FITC Labeling Kit: ab288089).

### Labeling Reaction:

1. Each vial of EZLabel FITC is sufficient for labeling of 1 mg of protein. One vial of EZLabel FITC was reconstituted with 10 μl of ethanol just before use.
2. For reconstitution, the substance was completely dissolved by pipetting up and down, followed by a dilution (1:5).
3. 100 μl of the prepared duramycin were transferred to a 1.5 ml microcentrifuge tube.
4. 10 μl reconstituted and diluted EZLabel FITC solution was added and mixed by pipetting up and down.
5. The mixed solution was incubated at RT on a rotary shaker or mixer for 1 hr.
6. After incubation, 20 μl EZLabel Quenching Buffer were added to quench the reaction. The reaction solution was incubated again at RT for 30 min.
7. Different fractions containing a single label on amino acid position 1, on amino acid position 2 and on both positions 1 and 2 were separated using HPLC.

### Analysis of Duramycin Fractions by Flow Cytometry:

Incubation of MCF-7 cell culture supernatant and RPMI cell culture medium without supplements (control) with 1% Triton (30 minutes; 400 rpm; 22° C.). Staining of samples with duramycin-FITC (1 ng/μl). Incubation for 30 minutes, 22° C., in the dark. Sample dilution 1:100 with 0.9% NaCl and analysis using flow cytometer (SA3800; Sony).

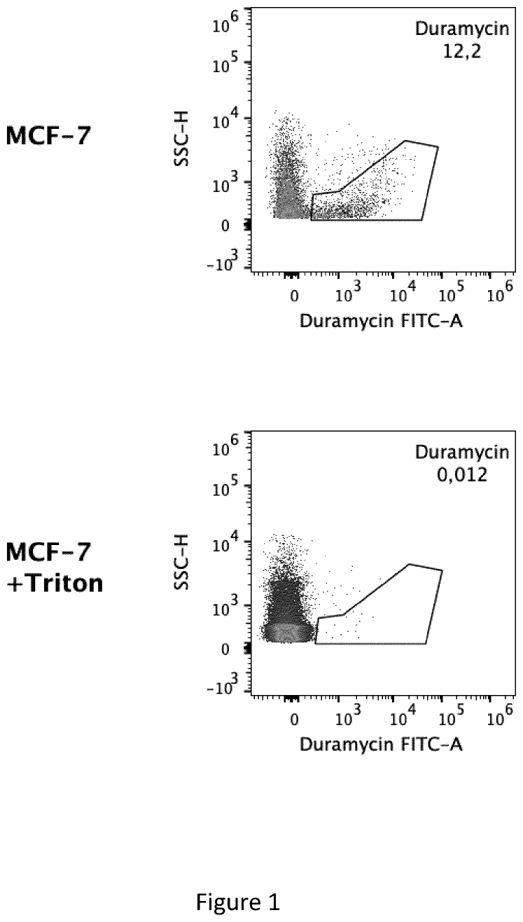

Results are shown in FIG. 1. Duramycin-FITC binds to exosomes in the cell culture supernatant of MCF-7 breast cancer cells (FIG. 1, upper panel). Triton treatment leads to destruction/solubilization of exosome, subsequently no binding occurs (FIG. 1, lower panel, negative control).

### Example 2

Labeling of duramycin with FITC as described in Example 1. MCF-7 supernatant and RPMI as control were pre-incubated with 1% Triton while shaking on a Thermomixer (1 h; 22° C.; 450 rpm). 10 μl of Triton-treated samples as well as 10 μl supernatant or RPMI were stained with different concentration of duramycin. Samples were incubated for 30 min at RT in the dark. Final sample dilution 1:100 with 0.9% NaCl. Measurement of samples using SA3800 flow cytometer.

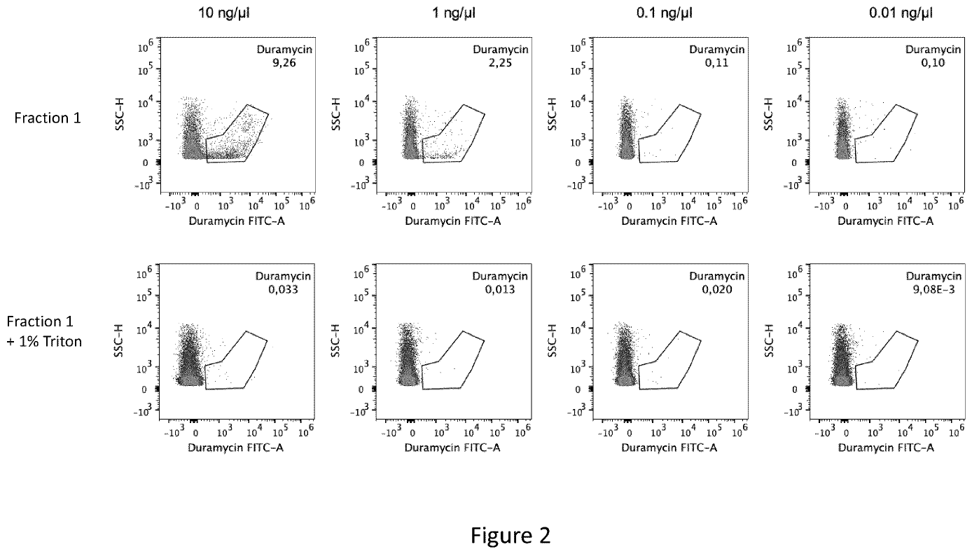

Results are shown in FIG. 2. Duramycin-FITC stains exosomes in cell culture supernatant of MCF-7 breast cancer cells in a concentration dependent manner (FIG. 2, upper panels). Triton treatment leads to destruction/solubilization of exosome, subsequently no binding occurs (FIG. 2, lower panels, negative control).

### Example 3

Labeling of duramycin with FITC as described in Example 1. Incubation of MCF-7 cell culture supernatant and RPMI medium (control) with 1% Triton (30 minutes; 400 rpm; 22° C.). Staining of samples with FITC-duramycin (1 ng/μl) and with CD9 PE antibody (1 ng/μl). Incubation for 30 minutes at 22° C. in the dark. Sample dilution 1:100 with 0.9% NaCl and analysis using flow cytometer (SA3800; Sony).

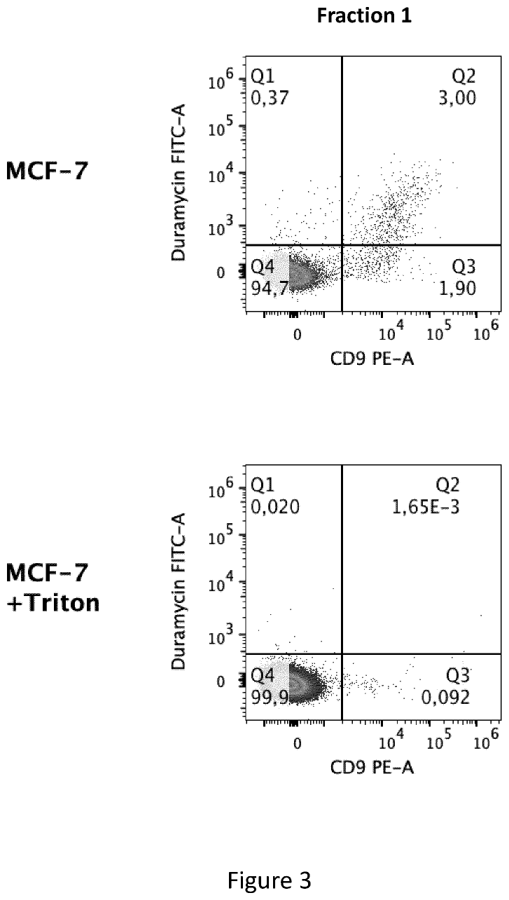

Results for double staining of exosomes with duramycin-FITC and the exosome marker CD9 are shown in FIG. 3 (upper panel). CD 9 and duramycin-FITC positive exosome population is depicted in the upper right quarter. Triton treatment leads to destruction/solubilization of exosome, subsequently no binding occurs (FIG. 3, lower panel, negative control, in which the upper right quarter is empty).

### Example 4

Labeling of duramycin with FITC as described in Example 1. Incubation of SKBR-3 (HER2 receptor positive cell line) cell culture supernatant or plasma and control medium with 1% Triton (1 hour; 500 rpm; 22° C.). Staining of samples with FITC-duramycin and HER2 antibody to perform double staining. Sample dilution 1:100 with 0.9% NaCl in case of cell culture supernatant; 1:1,000 with 0.9% NaCl in case of plasma. Sample analysis using flow cytometry (SA3800; Sony).

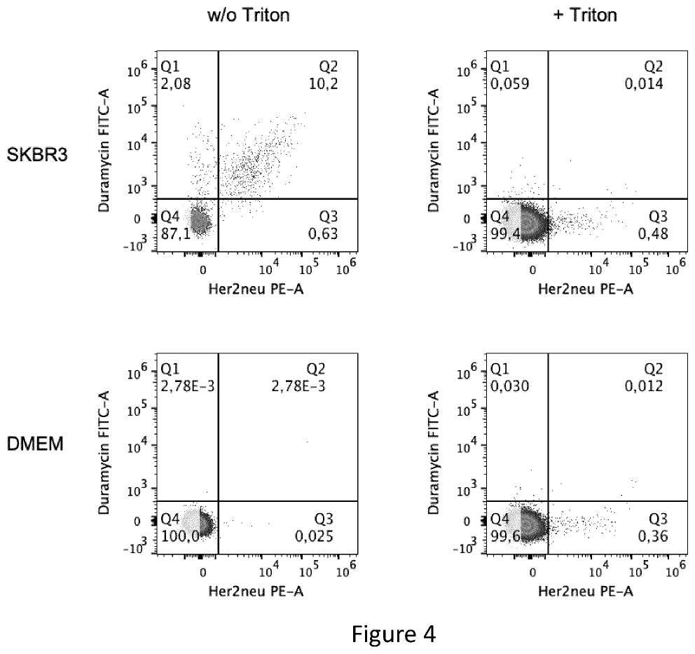

Results are shown in FIG. 4. HER2 positive exosomes double stained with HER2 antibody and FITC-duramycin (FIG. 4, upper left panel, upper right quadrant). To be noted, some exosomes did stain with FITC-duramycin but not with HER2 antibody (FIG. 4, upper left panel, upper left quadrant), indicating an exosome subpopulation which does not carry HER2 on its surface. If treated with Triton which solubilizes the exosomes, no signal is detected (FIG. 4, upper right panel). No staining is also observed in medium alone (DMEM) and also not in DMEM treated with Triton (FIG. 4 lower left and right panel, respectively).

### Example 5

Labeling of duramycin with FITC as described in Example 1. In order to generate EVs containing high levels of SARS-CoV-2 Spike^{FL} (D614G), a constitutive Spike expressing HEK293 cell line (CID4618, Helmholtz Zentrum München) was generated, and transfected with a S expression plasmid (#7413, Helmholtz Zentrum München) prior to the isolation of EVs from conditioned media. EVs were purified and concentrated by ultrafiltration, density gradient ultra-centrifugation and subsequently characterized by WB, NTA, vesicle flow cytometry and ELISA.

Incubation of HEK293-Spike1⁺ cell culture supernatant and HEKSN (HEK293 supernatant containing exosomes of unmodified cells) with 1% Triton (1 hour; 500 rpm; 22° C.). Staining of samples with FITC-duramycin or with Spike1 specific monoclonal antibody 35B12 to perform stainings. Sample dilution 1:100 with 0.9% NaCl in case of cell culture supernatant; 1:1,000 with 0.9% NaCl in case of plasma. Sample analysis using flow cytometry (exosome settings; SA3800; Sony).

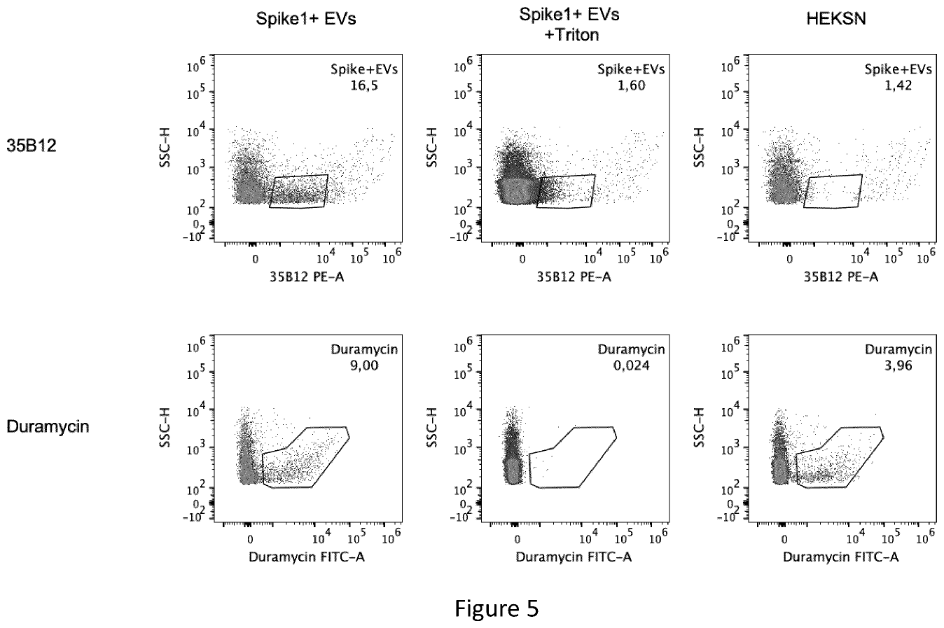

Results are shown in FIG. 5. SARS-CoV-2 Spike antibody stains exosomes from HEK293 Spike⁺ exosomes, Triton treatment reduces significant staining (Triton solubilizes exosomes), and HEK unmodified cells, Spike 1 negative, no staining is observed (FIG. 5, upper panels). FITC-duramycin stains exosomes in unmodified and Spike1⁺ exosomes, no staining in Triton treated exosomes (FIG. 5, lower panels).

### Example 6

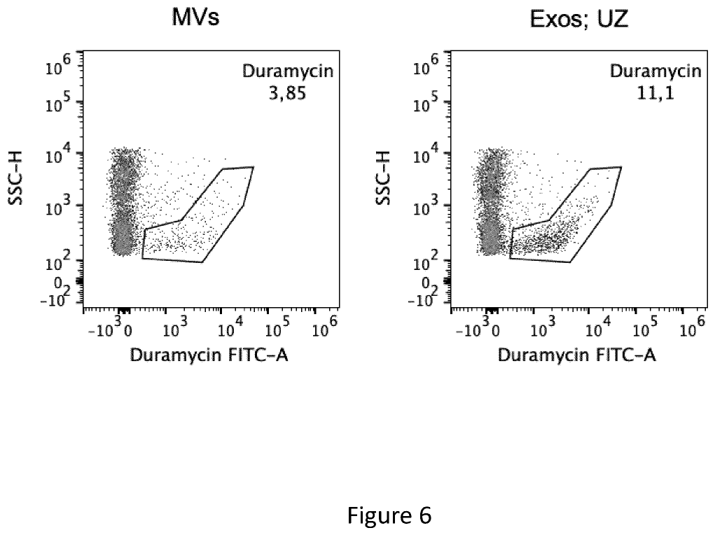

Labeling of duramycin with FITC as described in Example 1. Enrichment of EVs using ultracentrifugation. In the first approach, MCF-7 cell culture supernatant was centrifuged twice: The first centrifugation was performed for 15 min. at 1,200×g and at 20° C. The supernatant was then centrifuged for 35 min. at 14,000×g and at 4° C. The resulting microvesicle pellet was resuspended in PBS and subjected to flow cytometry analysis (FIG. 6, left panel; MV: microvesicle fraction). In the second approach, the supernatant of the second centrifugation step of the first approach was subjected to ultracentrifugation for 90 min. at 100,000×g and at 10° C. The pellet was resuspended in PBS and subjected to flow cytometry analysis (FIG. 6, right panel; Exo, UZ: exosome fraction).

The samples of both approaches were stained with FITC-labeled duramycin and signal intensity was compared using flow cytometry.

Results are shown in FIG. 6. The signal intensity was low in the microvesicle fraction (MV) when samples were centrifuged twice (left panel). An additional ultracentrifugation of the supernatant purifies exosomes, which increased the FITC-duramycin signal intensity in the flow cytometry analysis significantly (right panel), indicating that FITC-duramycin stains exosomes to a significantly larger degree than other microvesicles.

### Example 7

Extracellular vesicles (EV) derived from MCF-7 cell culture supernatant were stained with FITC-duramycin and CD9-PE. While duramycin stains all vesicles, the tetraspanin CD9 is not expressed on the surface of all cells, consequently not all vesicles are positive for CD9. The stained sample was subjected to flow cytometry and analyzed using a dot plot and the individual populations are identified using gating. In theory, up to three populations can be distinguished during sorting: CD9-PE single positive vesicles (CD9⁺), duramycin-single positive vesicles (duramycin⁺), and CD9⁺duramycin⁺ double positive vesicles. A “two-way sort” was carried out, i.e. sorting only for two populations: CD9⁺ single-positive vesicles and CD9⁺duramycin⁺ double-positive vesicles. The sorted samples were analyzed again using flow cytometry (SA3800; Sony). Here, the authenticity of the signal was checked by pre-incubating the sample with Triton before flow cytometry analysis, which solubilizes the EVs, leading to a signal reduction. If the sorted events are real vesicles, the signal can no longer be detected after Triton incubation.

Analysis of CD9-PE signal in all samples revealed a high amount of CD9⁺ counts/ml in the double positive sorted samples which was resolved by triton treatment. Samples sorted only for CD9⁺ signal showed approximately similar counts/ml compared to triton treated samples, suggesting antibody aggregates.

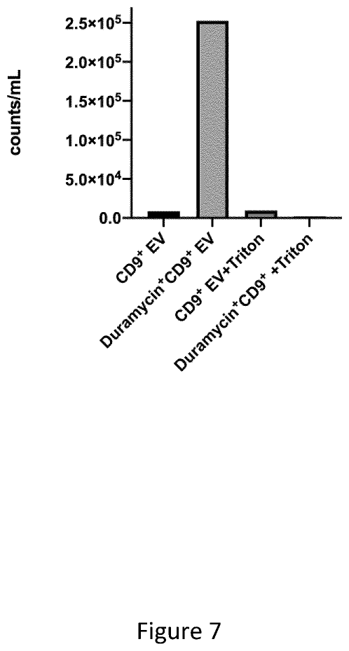

FIG. 7 shows that exosomes were detected with FITC-duramycin which were also CD9-PE positive. There were no significant amounts of exosomes detected which are only CD9-PE positive and not duramycin positive. Triton treatment, which solubilizes the exosomes, did not lead to any (significant) detection (negative control), demonstrating the specificity of the results. A detailed description of the experimental set-up is provided in the following.

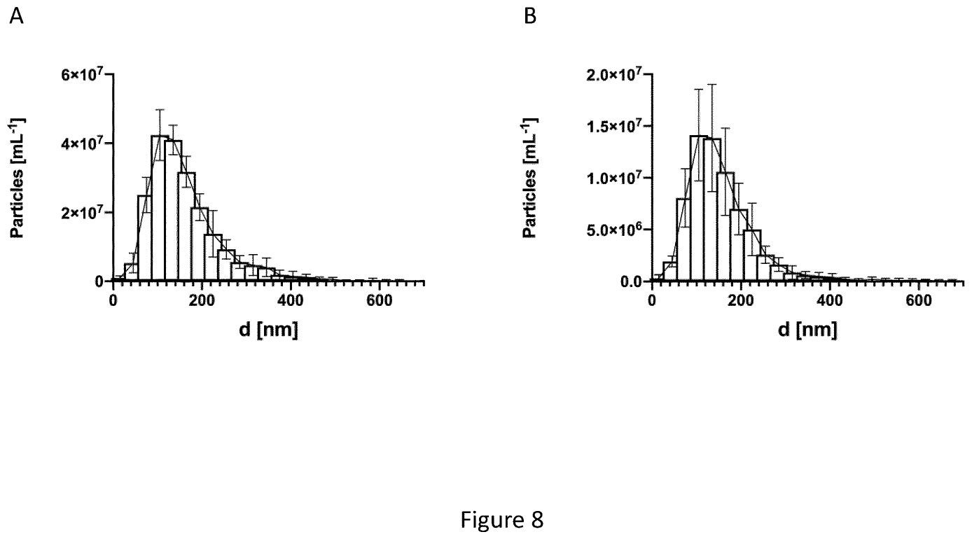

The sorted samples were diluted in filtered PBS and size distribution analysis was performed applying nanoparticle tracking analysis (NTA) using the ZetaView PMX110 from Particle Metrix. Each sample was measured six times as technical replicate. Graphs show size distribution of duramycin⁺ sorted vesicles (FIG. 8A) as well as duramycin⁺ CD9⁺ sorted vesicles (FIG. 8B), which show an average diameter within the range of between 75 nm and 200 nm in both samples.

1) Preparation of EVs from MCF-7 Cell Culture Supernatant

The complete culture medium (RPMI+10% FCS+1% Penicillin/Streptomycin (Pen/Strep)) was removed from the cells and replaced by RPMI medium without supplements. 24 hours after medium exchange, the cell culture supernatant was removed from cells. While the complete medium was again added to cultivated cells, removed cell culture supernatant (10 ml in total) was centrifuged at 2,000×g at 21° C. for 15 minutes. The supernatant was transferred into 2 ml reaction tubes and centrifuged for 10 minutes at 10,000×g and 4° C.

### 2) Staining of Samples for Sorting

10 ml of the MCF-7 cell culture supernatant was divided into two samples (2×5 ml). Sample 1 contained 5 ml of the MCF-7 cell culture supernatant stained with 100 ng/μl FITC-duramycin (Batch 9) and CD9 PE (1 ng/μl). Sample 2 contained 5 ml of the MCF-7 cell culture supernatant stained with 100 ng/μl FITC-duramycin (Batch 9). Both samples were incubated for 30 minutes in the dark at room temperature. Three falcons with 100 μl PBS (PAN Technology) were prepared.

### 3) Sorting

Sorting was performed using the Astrios Sorter (MoFLo) from Beckman Coulter. Samples were sorted in “purify mode”.
Sample 1 (stained for FITC-duramycin and CD9 PE):
   Falcon 1: Sort for CD9 PE⁺ events
   Falcon 2: Sort for FITC-duramycin⁺ CD9 PE⁺ events
Sample 2 (stained for FITC-duramycin):
   Falcon 3: Sort for FITC-duramycin⁺ events
      Results are shown in the table below:

| | | |
|---|---|---|
| Falcon 1 | Sort for CD9-PE⁺ events | 3.5 × 10⁴ (140 μl in |
| | | falcon after sort) |
| Falcon 2 | Sort for FITC-duramycin⁺ | 4.59 × 10⁵ (480 μl in |
| | CD9-PE⁺ events | falcon after sort) |
| Falcon 3 | Sort for FITC-duramycin⁺ | 2.6 × 10⁴ (122.6 μl in |
| | events | falcon after sort) |

### 4) Triton Treatment

Sorted samples as well as unsorted samples were incubated with Triton. 18 μl of each sorted sample were incubated with 2 μl 10% Triton or as control with 2 μl 0.9% NaCl for 1 hour on a shaker (500 rpm) at room temperature (22° C.). For the unsorted samples, 45 μl of the MCF-7 cell culture supernatant were incubated with 5 μl 10% Triton for 1 hour on a shaker (500 rpm) at room temperature (22° C.).

5) Staining of Samples with Antibodies

20 μl of the non-sorted samples were stained with 100 ng/μl FITC-duramycin and CD9 PE (1 ng/μl) as single and double stains. 20 μl FITC-duramycin⁺ CD9 PE⁺ sorted samples were not stained; CD9 PE⁺ sorted sample was stained for FITC-duramycin (100 ng/μl); FITC-duramycin⁺ sorted sample was stained with CD9 PE (1 ng/μl). The samples were incubated for 30 minutes at room temperature in the dark. Non-sorted samples were finally diluted 1:100 with 0.9% NaCl in a 2-step process: 90 μl 0.9% NaCl was added to each sample, then the samples were resuspended, and 20 μl were added to 180 μl of 0.9% NaCl and counting beads (Beads/50p: 0.5×10⁵ beads). Sorted samples were only diluted 1:10 with 0.9% NaCl by adding 180 μl NaCl and Counting Beads to each sample. Sample measurement was performed using the flow cytometer SA3800 (Sony).

### 6) NTA (Nano Tracking Analysis) Measurement

All measurements were performed in a total volume of 1 ml. The following samples were measured with the Zetaviewer from Particle Metrix.
a) PBS buffer (137 mM Sodium Chloride, 2.7 mM Potassium Chloride and 12 mM Phosphate-buffer, pH 7.4 (PAN Biotec)); not diluted
b) RPMI serum without supplements: not diluted
c) MCF-7 cell culture supernatant (not sorted); diluted 1:5 in PBS
d) Sorted samples (each sample was adjusted to 1 ml in total with PBS
   CD9 PE⁺: 100 μl sample+900 μl PBS
   FITC-duramycin⁺: 82.6 μl sample+917.4 μl PBS
   CD9 PE⁺ FITC-duramycin⁺: 400 μl sample+600 μl PBS
      Each sample was measured 6 times to calculate a mean. Results are shown in FIGS. 7 and 8.

### Example 8

Labelling of Exosomes from Plasma and ApoE Staining

Blood plasma was obtained from healthy donors 1, 3 and 4 hours after having fatty food, as well as from fastened donors. Serum samples were prepared by centrifuging serum samples at 2,000×g for 15 minutes at 20° C. Plasma samples were obtained by centrifuging samples at 2,000×g for 15 minutes at 20° C.; transferring the samples into new reaction tubes, centrifuging the samples at 2,000×g for 15 minutes at 20° C.; transferring the samples into 2 ml reaction tubes, and centrifuging the 2 ml reaction tubes at 10,000×g for 10 minutes at 4° C.

The following table shows the detection agents used for staining.

| Detection agents | Fluorochrome | stock conc. | | Cat. no | Lot. no | Company | Pre-dilution |
|---|---|---|---|---|---|---|---|
| CD41 | PB | 80 | μg/ml | 303714 | B264793 | Biolegend | 1:8 |
| ApoE | PE | 50 | μg/ml | 803404 | | Biolegend | 1:5 |
| ELAEXIA | FITC | 1 | mg/ml | Bt. 13 | — | Self-labeled | 1:10 |

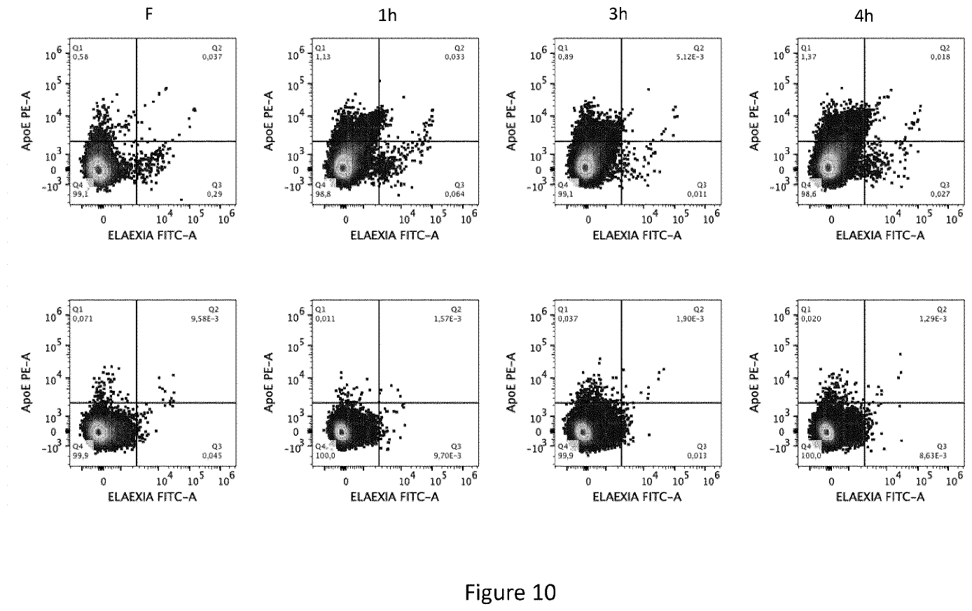

All plasma samples were pre-diluted 1:10 followed by a Triton incubation step for one hour: centrifugation at 450 rpm at 21° C. of 90 μl of pre-diluted plasma+10 μl 10% Triton or 10 μl NaCl. The samples were stained with 1 ng/μl ApoE in combination with 10 ng/μl ELAEXIA (Elective Labelling of Exosomes for Identification and Analysis; in this example duramycin and FITC) and incubated for 30 minutes at 21° C. in the dark. Subsequently, samples were diluted 1:100 resulting in a final 1:1,000 dilution of plasma (1:100 dilution in two steps; NaCl in second step includes counting beads to quantify the exosomes (Count Bright Absolute Counting Beads, ThermoFisher, Cat. No.: #C36950, Lot.: 2466363). FIG. 10 shows the results analyzed by flow cytometry (F=fastened; 1 h, 3 h, 4 h=1 hour, 3 hours and 4 hours, respectively after fatty food intake). Upper panel shows ApoE and ELAEXIA staining of plasma at the fastened state, and 1, 3 and 4 hours after food intake. ELAEXIA positive exosomes are not stained with ApoE (right upper quadrant is empty), lower panel shows the negative control, i.e. samples treated with Triton to solubilize the exosomes.

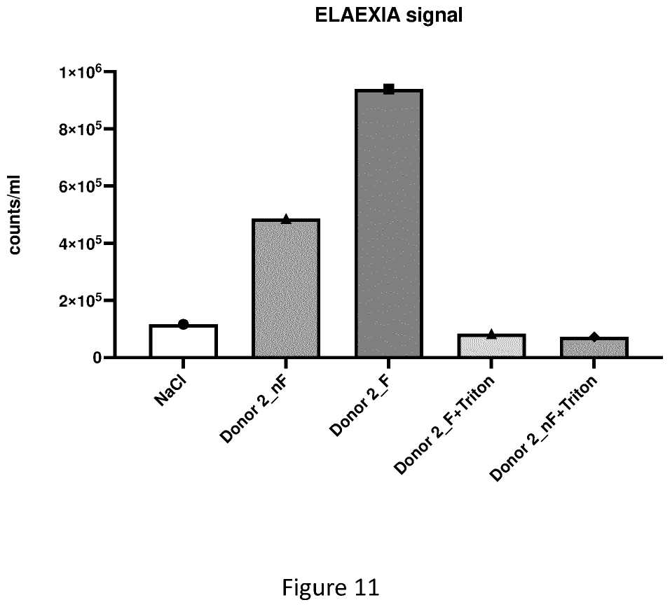

FIG. 11 shows ELAEXIA staining of plasma of one healthy donor at fastened state and after food intake (non-fastened). NaCl=negative control staining; Donor 2_nF=ELAEXIA staining of exosomes at non-fastened state; Donor 2_F=ELAEXIA staining of exosomes at fastened state; Donor 2_nF+Triton and Donor 2_F+Triton=negative control with the same samples but pretreated with Triton to solubilize the exosomes, which interferes with staining.

### Example 9

### Cinnamycin Staining of Exosomes

Labeling of cinnamycin with FITC as described in Example 1. Samples were prepared from supernatant of MCF-7 cells cultured in RPMI. Preparation of MCF-7cell culture supernatant containing EVS was performed by removing the complete medium (RPMI+supplements+10% FCS+1% Pen/Strep) from the cells and replacing it with RPMI medium without any supplements. 24 hours after medium exchange, cell culture supernatant was removed from cells. The removed cell culture supernatant was centrifuged at 2,000×g at 21° C. for 15 minutes. Centrifuge supernatant was transferred into 2 ml reaction tubes and centrifuged for 10 minutes at 10,000×g at 4° C. 225 μl of cell culture supernatant or cell culture medium was incubated with 25 μl 10% Triton for 1 hour on a shaker (500 rpm) at room temperature (22° C.). Samples were stained as follows: Cinnamycin Batch: 1 ng/μl or 10 ng/μl; CD9 PE: 1 ng/μl. For staining, samples were incubated for 30 minutes at 22° C. in the dark and diluted 1:100 before flow-cytometry measurement using the flow cytometer SA3800 (Sony, exosome settings).

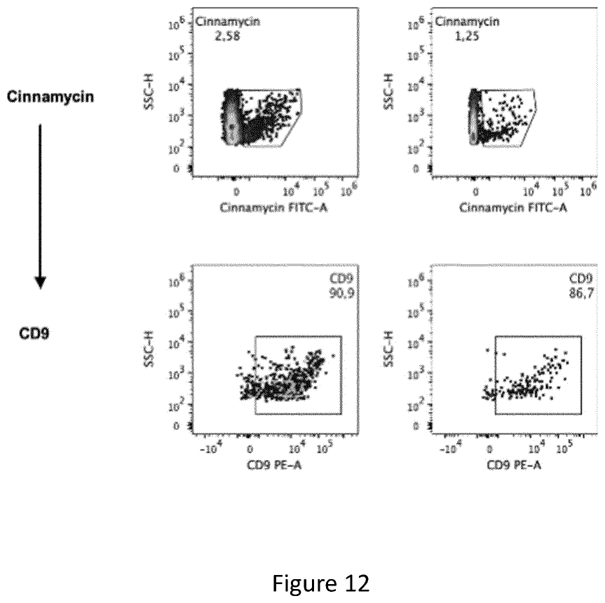

FIG. 12 shows the results for staining with cinnamycin labelled with FITC (ELAEXIA) (FIG. 12 upper panel; 10 ng/ml cinnamycin left panel, 1 ng/ml cinnamycin right panel). Subsequently, cinnamycin positive stained exosomes were additionally stained with anti-CD9 antibody to show that the method purifies exosomes, since exosomes of cell line MCF-7 are CD9+(FIG. 12, lower panel). The results show that fluorescently labelled cinnamycin stains exosomes to a similar extend as fluorescently labelled duramycin.

### LITERATURE

Barroso M M. Quantum Dots in Cell Biology; J. Histochem. Cytochem., 2011. Vol. 59(3): 237-251
Boing A N, van der Pol E, Grootemaat A E, Coumans F A, Sturk A, Nieuwland R (2014). Single-step isolation of extracellular vesicles by size-exclusion chromatography. Journal of Extracellular Vesicles. 3: 23430
Blanc L and Vidal M. New insights into the function of Rab GTPases in the context of exosomeal secretion. Small GTPases 2018. Vol. 9, NOS 1-2, 95-106.
Brennetta J. Crenshaw, Linlin Gu, Brian Sims and Qiana L. Matthews1, Exosome Biogenesis and Biological Function in Response to Viral Infections. The Open Virology Journal DOI: 10.2174/1874357901812010134, 2018, 12, 134-148
Gebara N, Scheel J, Skovronova R, Grange C, Marozio L, Gupta S, Giorgione V, Caicci F, Benedetto C, Khalil A, Bussolati B. Single extracellular vesicle analysis in human amniotic fluid shows evidence of phenotype alterations in preeclampsia. Journal of Extracellular Vesicles, 2022. 11(5): e12217
Harding C, Heuser J, Stahl P. Receptor-mediated endocytosis of transferrin and recycling of the transferrin receptor in rat reticulocytes. J. Cell Biol. 1983; 97:329-339
Hench I B, Hench J, Tolnay M. Liquid Biopsy in Clinical Management of Breast, Lung, and Colorectal Cancer. Frontiers in Medicine 2018. Vol. 5, Article 9
Hullin-Matsuda F, Makino A, Murate M, Kobayashi T. Probing phosphoethanolamine-containing lipids in membranes with duramycin/cinnamycin and aegerolysin proteins. Biochimie. 2016 November; 130:81-90. doi: 10.1016/j.biochi.2016.09.020. Epub 2016 Sep. 28. PMID: 27693589.
Jia et al. Exosome: emerging biomarker in breast cancer. Oncotarget. 2017; 8:41717-41733
Johnstone R M, Bianchini A, Teng K. Reticulocyte maturation and exosome release: transferrin receptor containing exosomes shows multiple plasma membrane functions. Blood 1989; 74: 1844-1851.
Leuenberger H G W, Nagel B and Kölbl H. eds. 1995. Helvetica Chimica Acta, CH-4010 Basel, Switzerland
Li P, Kaslan M, Lee S H, Yao J and Gao Z. Progress in Exosome Isolation Techniques. Theranosics 2017. Vol. 7 (3): 789-804
Makino A, Baba T, Fujimoto K, Umeda M, Matsuzaki K, Kobayashi T. Cinnamycin (Ro 09-0198) Promotes Cell Binding and Toxicity by Inducing Transbilayer Lipid Movement. J Biol Chem. 2003, 278(5): 3204-3209
McAndrews K and Kallun R. Mechanisms associated with biogenesis of exosomes in cancer. Molecular Cancer 2019. DOI.org/10.1186/s12943-019-0963-9
Neice A. Chapter 3—Methods and Limitations of Subwavelength Imaging. Advances in Imaging and Electron Physics 2010. Vol. 163:117-140.
Navarro J, Chabot J, Sherrill K, Aneja R, Zahler A S, Racker E. Interaction of duramycin with artificial and natural membranes. Biochemistry. 1985 Aug. 13, 24(17):4645-4650
Pan B T, Johnstone R M. Fate of the transferrin receptor during maturation of sheep reticulocytes in vitro: selective externalization of the receptor. Cell 1983; 33:967-978
Savina A, Vidal M, Colombo M I. The exosome pathwayin K562 cells is regulated by Rab11. J Cell Sci 2002; 115:2505-15; PMID:12045221
Stockert J C, Blázquez-Castro A. Chapter 20—Super-resolution Microscopy. Fluorescence Microscopy in Life Sciences 2017. Bentham Science Publishers. pp. 687-711.
Subra C, Laulagnier K, Perret B, Record M. Exosome lipidomics unravels lipid sorting at the level of multivesicular bodies. Biochimie 2007 February; 89(2):205-212
Tauro B J, Greening D W, Mathias R A, Ji H, Mathivanan S, Scott A M, Simpson R J (February 2012). “Comparison of ultracentrifugation, density gradient separation, and immunoaffinity capture methods for isolating human colon cancer cell line LIM1863-derived exosomes”. Methods. 56 (2): 293-304
Thind A, Wilson C (2016). “Exosomal miRNAs as cancer biomarkers and therapeutic targets”. Journal of Extracellular Vesicles. 5: 31292
van der Pol E, Boing A N, Harrison P, Sturk A, Nieuwland R (July 2012). “Classification, functions, and clinical relevance of extracellular vesicles”. Pharmacological Reviews. 64 (3): 676-705
Van Deun J, Mestdagh P, Sormunen R, Cocquyt V, Vermaelen K, Vandesompele J, Bracke M, De Wever O, Hendrix A (2014). “The impact of disparate isolation methods for extracellular vesicles on downstream RNA profiling”. Journal of Extracellular Vesicles. 3: 24858
Vidal M, Mangeat P, Hoekstra D. Aggregation reroutesmolecules from a recycling to a vesicle-mediated secre-tion pathway during reticulocyte maturation. J Cell Sci 1997; 110(16):1867-77; PMID:9296387
Widdick et al. 2003 https://doi.org/10.1073/pnas.0230516100
Zhao M, Li Z, Bugenhagen S. ^{99m}Tc-Labeled Duramycin as a Novel Phosphatidylethanolamine-Binding Molecular Probe. J Nucl Med. 2008, 49: 1345-1352

1. A method for detecting and/or quantifying exosomes in a sample, the method comprising the steps of:
i) providing a sample that is known to comprise or suspected of comprising exosomes;
ii) contacting a binding agent that specifically binds to the membrane of extracellular vesicles with the sample; and
iii) detecting and/or quantifying exosomes by flow cytometry and/or super-resolution microscopy, and optionally isolating exosomes;
wherein the binding agent comprises a label and a phosphatidylethanolamine-binding lantibiotic,
wherein the label is preferably selected from the group consisting of an enzyme label, a fluorochrome, a peptide label, a radioactive label, a ligand, a magnetic or paramagnetic label, and a quantum dot, and
wherein the lantibiotic is preferably selected from the group consisting of duramycin and cinnamycin.

2. The method of claim 1, wherein the lantibiotic is cinnamycin.

3. The method of claim 1, wherein:
(a) the binding agent is bound by a further binding agent specifically binding to the binding agent or to its label; or
(b) the binding agent is bound to a solid surface.

4. The method of claim 1, wherein the sample is a cell culture supernatant, a cell preparation obtained by lysing and/or centrifuging the cells, or a body fluid.

5. The method of claim 4, wherein the body fluid is selected from the group consisting of plasma, serum, ascites, cerebral fluid, bone marrow, urine, faeces and bronco-alveolar washing.

6. The method of claim 1, wherein the method further comprises prior to step i) one or more of the following steps:
a) suspending and/or solubilizing the sample;
b) ultrafiltration and/or centrifuging the sample; and/or
c) enriching the exosome concentration in the sample by size- and/or density-centrifugation.

7. The method according to claim 1,
wherein the sample is obtained from a subject known or suspected to suffer from a disease, wherein the method further comprises comparing the quantity of exosomes in the sample of the subject known or suspected to suffer from a disease with the quantity of exosomes known to be present in a sample of a healthy subject, wherein an increase in the quantity of exosomes in the sample of the subject known or suspected to suffer from a disease is indicative of the presence or stage of the disease,
preferably wherein an increase in the quantity of exosomes in the sample of the subject known or suspected to suffer from a disease is indicative of the presence or stage of cancer.

8. The method of claim 7, wherein comparing the quantity of exosomes comprises applying CD mapping and t-SNE analysis, NTA Tracking, Zetasizer and applying counting beads with flow cytometry.

9. The method according to claim 1, further comprising analyzing surface markers and/or the content of the exosomes,
preferably wherein the surface markers and/or the content of the exosomes comprise one or more of peptides, proteins, microRNA, DNA, and/or RNA, more preferably wherein analyzing the content of the exosomes comprises DNA mutation analysis, RNA expression, DNA methylation quantification and/or protein expression.

10. A method for diagnosing cancer, comprising:
a) detecting and isolating exosomes from a sample by the method of claim 1, and
b) detecting within the isolated set of exosomes obtained in step a) those exosomes that present cancer antigens.

11. A method for detecting or diagnosing a virus disease, comprising:
a) detecting and isolating exosomes from a sample by the method of claim 1, and
b) detecting within the isolated set of exosomes obtained in step a) those exosomes that present a viral antigen,
preferably wherein the viral antigen is a virus surface protein, more preferably a spike protein, most preferably a spike protein of the SARS-CoV-2 or SARS-CoV-1 virus or of any other virus.

12. A method for quantifying and/or qualifying tumor-related exosomes in a sample, said method comprising the steps of:
a) detecting and isolating exosomes from a sample by the method of claim 1; and
b) detecting within the isolated set of exosomes obtained in step a) those exosomes that are tumor-related by using at least one binding agent specifically binding to a tumor antigen.

13. A method for monitoring tumor growth, said method comprising the step of:
periodically quantifying the number of tumor related exosomes in a sample with the method according to claim 12, wherein an increase in the number of tumor related exosomes between two quantifications indicates tumor growth.

14. A method for monitoring a virus disease, said method comprising the step of:
periodically quantifying the number of virus-related exosomes in a sample with the method according to claim 1.

15. A kit comprising:
(i) a binding agent comprising a label and a phosphatidylethanolamine-binding lantibiotic,
wherein the label is preferably selected from the group consisting of an enzyme label, a fluorochrome, a peptide label, a radioactive label, a ligand, a magnetic label, or a quantum dot, and wherein the lantibiotic is preferably selected from the group consisting of duramycin and cinnamycin, more preferably wherein the lantibiotic is cinnamycin; and
(ii) instructions for binding of said binding agent to exosomes in a sample and for applying flow cytometry and/or super resolution microscopy for detecting, quantifying, enriching or producing exosomes.


SEQUENCE LISTING (4 sequences)

SEQ ID NO: 1 - protein, 19 aa, Streptoverticillium cinnamoneus
  SITE 11: note = X is alpha-aminobutyric acid
  SITE 18: note = X is alpha-aminobutyric acid
AKQAAAFGPFXFVADGNXL

SEQ ID NO: 2 - protein, 19 aa, Streptoverticillium cinnamoneus
  SITE 11: note = X is alpha-aminobutyric acid
  SITE 18: note = X is alpha-aminobutyric acid
ARQAAAFGPFXFVADGNXL

SEQ ID NO: 3 - protein, 8 aa, synthetic construct
WSHPQFEK

SEQ ID NO: 4 - protein, 28 aa, synthetic construct
WSHPQFEKGGGSGGGSGGSAWSHPQFEK